(19) United States
(12) Patent Application Publication
(10) Pub. No.: US 2012/0220788 A1
(43) Pub. Date: Aug. 30, 2012

(54) Continuous preparation of carbonates

(75) Inventor: Alain Lambert, Beauvechain (BE)

(73) Assignee: SOLVAY FLUOR GMBH, Hannover (DE)

(21) Appl. No.: 13/497,309

(22) PCT Filed: Sep. 27, 2010
(86) PCT No.: PCT/EP2010/064218

§ 371 Date: Mar. 21, 2012

(30) Foreign Application Priority Data
Sep. 28, 2009 (EP) 09171489.9

(51) Int. Cl. C07D 317/42; C07C 69/96

(52) U.S. Cl. 549/229; 558/260

(57) ABSTRACT

A process for the manufacture of fluoroethylene carbonate or difluoroethylene carbonate by reaction of ethylene carbonate and F₂ or for the manufacture of fluoromethyl methyl carbonate or difluorinated dimethyl carbonate by reaction of dimethyl carbonate and F₂ is described wherein such fluorination process is performed continuously.

### CROSS-REFERENCE TO RELATED APPLICATIONS

The present application is a U.S. national stage entry under 35 U.S.C. §371 of International Application No. PCT/EP2010/064218 filed Sep. 27, 2010, which claims benefit of European patent application number 09171489.9 filed on Sep. 28, 2009, the complete content of this application being incorporated herein by reference for all purposes.

### TECHNICAL FIELD OF THE INVENTION

The present invention concerns a process for the continuous preparation of certain fluorosubstituted organic carbonates.

### BACKGROUND

Monofluoroethylene carbonate and fluoromethyl methyl carbonate as well as difluoroethylene carbonate and difluorinated dimethyl carbonate are especially suitable as solvents or solvent additives for lithium ion batteries. Monofluoroethylene carbonate can be prepared from the respective unsubstituted ethylene carbonate by the reaction of 1,3-dioxolane-2-one (ethylene carbonate; “EC”) with elemental fluorine. This is described for example in JP-A 2000-309583 where the reaction is performed with a melt of EC or its solution in anhydrous fluoride. Optionally, perfluorohexane can be present; in this case, a suspension of 1,3-dioxolane-2-one—the starting material—is formed. According to US patent application 2006-0036102, ethylene carbonate is dissolved in FlEC (Fluoroethylene carbonate) and then is contacted with diluted fluorine. According to U.S. Pat. No. 7,268,238, the reaction is performed in a reactor with Raschig rings to provide a suitable bubble size of the diluted fluorine gas. According to the state of the art, the fluorination reactions and the isolation of the product were performed in batch processes.

### SUMMARY OF THE PRESENT INVENTION

Subject of the present invention is to provide a process for the preparation of fluorosubstituted organic carbonates selected from the group consisting of fluoroethylene carbonate, fluoromethyl methyl carbonate, difluoroethylene carbonate and difluorinated dimethyl carbonate in a technically feasible manner with good yield and selectivity.

The present invention provides a liquid phase process for the manufacture of an organic carbonate selected from the group consisting of fluoroethylene carbonate, difluoroethylene carbonate, fluoromethyl methyl carbonate and difluorinated dimethyl carbonate by reaction of ethylene carbonate and diluted F₂ to produce fluoroethylene carbonate or difluoroethylene carbonate and by the reaction of dimethyl carbonate with diluted F₂ to produce fluoromethyl methyl carbonate or difluorinated dimethyl carbonate wherein the process is performed continuously. In the process of the present invention, the diluted fluorine is dispersed in gaseous form into the liquid carbonate. Thus, the process of the invention is a 2-phase process. Fluorine is introduced in diluted form to improve the safety of the process, and because a lot of reaction heat is generated which will be too high if pure fluorine would be applied.

The term “continuously” is understood to denote a continuous introduction of diluted fluorine and a continuous introduction of ethylene carbonate or dimethyl carbonate. If a single reactor is applied which contains several compartments (considered as cascade) connected to each other, for example, via perforated plates, one would expediently introduce diluted fluorine gas into the bottom compartment of the reactor, and carbonate at the top compartment. If a cascade of separate reactors is applied, diluted fluorine and liquid carbonate are usually introduced continuously in each reactor. The reaction can be performed in a single reactor.

A single reactor can be used which has one reaction compartment, but selectivity is low due to consecutive fluorination steps. It is also possible to perform the reaction in a single reactor which has 2 or more compartments arranged one above the other which compartments are separated for example by perforated plates which reduce the mass transfer of the reaction mixture, but allow the passage of fluorine gas through the compartments. In a preferred embodiment, the reaction is performed in a cascade of 2 or more reactors. More reactors provide improved selectivity and conversion, but raise the costs. A reactor cascade comprising 2 to 5 reactors is highly suitable. Cascades with 2, 3, and 4 reactors are preferred, and cascades with 2 or 3 reactors are most preferred. Fluorine gas (or, preferably, a mixture of fluorine gas and nitrogen or other inert gas) is introduced into any reactor of the cascade. If desired, the reactors are assembled in a single reactor in the form of separate compartments, e.g. in one reactor with 2, 3, 4 or 5 partition plates or with means having the same effect.

Elemental fluorine is applied in diluted form. Preferred diluents are inert gases, especially inert gases selected from the group consisting of nitrogen, noble gases or mixtures thereof. A mixture of elemental fluorine and nitrogen is preferred. The concentration of fluorine is greater than 0% by volume. It is preferably equal to or greater than 5% by volume. It is more preferably equal to or greater than 12% by volume. The concentration of fluorine is preferably equal to or lower than 25% by volume. Preferably, it is equal to or lower than 18% by volume. Preferably, fluorine is contained in the gas mixture in a range of 12 to 18% by volume. While it is possible to introduce into the different reactors different gas mixtures with different concentrations of fluorine or with different inert gases, or diluted and undiluted fluorine gas, it is preferred for practical reasons to apply only one specific gas or gas mixture for all reactors.

In the following, the term “fluorine” is understood to denote fluorine diluted by inert gas, notably diluted by nitrogen.

It is preferred to introduce the fluorine into the liquid as finely dispersed bubbles. If fluorine is introduced in finely dispersed form, a high contact surface is provided. A good dispersion of the gas can be achieved by passing it through a frit made of material resistant to fluorine and HF. Frits made from stainless steel, alloys which are resistant to fluorine and HF like Monel, Inconel or Hastelloy, or perfluorinated polymeric material, e.g. polytetrafluoroethylene, are preferred. The gas bubbles generate a sufficient mixing of the reaction mixture in the reactor. If desired, the reaction can be performed in continuous stirred reactors (“CSTR”).

Since the fluorination reaction generates a lot of heat, it is mandatory to cool the reaction mixture to perform the reaction effectively.

Cooling of the reaction mixture is done in a manner known in the art. For example, the reactor or reactors might have cooling jackets or internal heat exchangers; but cooling is very poor. The use of external coolers for cooling of the reaction mixture is preferred. Preferably, a part of the reaction mixture is continuously withdrawn from the reactor and flows through an external cooler before returning back to the reactor. The continuous circulation of a part of the reaction mixture for cooling purposes improves the mixing of the reaction mixture.

The reaction mixture contains hydrogen fluoride which is a reaction product. Generally, the content of HF will be in the range of about 1 to about 10% by weight of the reaction mixture. The HF concentration, depends on the temperature of the reaction mixture, the pressure, the amount of nitrogen in the F₂/N₂ mixture (or of the content of other inert gas), the gas/liquid mass transfer conditions and, especially, on the conversion of starting carbonate which is related to the molar ratio of F₂ and the carbonate fed to the reactor.

According to one embodiment, the reaction is performed to produce the monofluorinated products, namely monofluoroethylene carbonate from ethylene carbonate, or fluoromethyl methyl carbonate from dimethyl carbonate. This embodiment is preferred. According to another embodiment, the reaction is performed to produce the difluorinated compounds, namely 4,4-difluoro-1,3-dioxolane-2-one, cis and trans-4,5-difluoro-13,dioxolane-2-one from ethylene fluoride, or difluoromethyl methyl carbonate and bis-difluoromethyl carbonate from dimethyl carbonate. In this embodiment, instead of ethylene carbonate, monofluoroethylene carbonate or a mixture of ethylene carbonate and monofluoroethylene carbonate can be applied as starting material. Likewise, instead of dimethyl carbonate, fluoromethyl methyl carbonate or a mixture of dimethyl carbonate and fluoromethyl methyl carbonate can be applied as starting material to produce difluorinated dimethyl carbonate. The term “difluorinated dimethyl carbonate” denotes fluoromethyl methyl carbonate and bis-fluoromethyl carbonate which, in the process of the present invention, are generated simultaneously.

### BRIEF DESCRIPTION OF THE DRAWING

For a detailed description of preferred embodiments of the invention, reference will now be made to the accompanying drawing, in which:

FIG. 1 illustrates a 2-reactor cascade in which the process of the invention can be performed.

### DETAILED DESCRIPTION OF THE PREFERRED EMBODIMENTS

The invention will now be described in detail in view of the preferred embodiment, the preparation of monofluoroethylene carbonate and fluoromethyl methyl carbonate.

The reaction may be performed at a temperature which is higher than the melting point of the starting material. Dimethyl carbonate melts at about 2 to 4° C., ethylene carbonate melts at about 34 to 37° C. If desired, the melting point can be lowered by using solvents inert towards fluorine and HF which is a reaction product. For example, HF could be used as solvent. Perfluorocarbons, for example, perfluorohexane or perfluorocyclohexane, can also be used. In a preferred embodiment, fluoroethylene carbonate is applied as solvent for ethylene carbonate, especially preferably in the start-up phase. In a preferred embodiment, ethylene carbonate and dimethyl carbonate starting material is introduced neat into the reaction. “Neat” means that the carbonate educt is undiluted and contains no inert solvent. In a preferred embodiment, no inert solvents are applied throughout the reaction; in this embodiment, the starting material is not introduced in the form of a mixture with an inert solvent, no inert solvent is added separately, and the reaction mixture also does not contain an inert solvent even in the start-up phase. The term “inert” denotes compounds which, under the reaction conditions of the fluorination and the isolation of the product, do not react substantially with fluorine or HF. Fluoroethylene carbonate and fluoromethyl carbonate are not considered inert. The term “substantially” denotes preferably that equal to less than 1% by weight of the inert solvent reacts with F₂ or HF during 1 hour of performing the reaction. Since during the continuous fluorination reaction there is always a certain level of fluoroethylene carbonate present in the reaction mixture, the addition of fluoroethylene carbonate to the reaction mixture would be of advantage only initially in the start-up phase. During the fluorination reaction, preferably no fluoroethylene carbonate is introduced in the reactor. The melting point of dimethyl carbonate is low enough so that no solvent would be needed, but if desired, a perfluorinated solvent or fluoromethyl methyl carbonate could be used as solvent.

Preferably, according to a first embodiment for the manufacture of fluoroethylene carbonate, the reaction temperature is equal to or higher than 40° C. Generally, the reaction temperature is equal to or lower than 100° C.; but at such a high temperature, fluorinated reaction product may be contained in gas streams leaving the reactor and must be recovered to prevent lowering of the yield. Preferably, the reaction temperature is equal to or lower than 80° C., more preferably, it is equal to or lower than 70° C., and most preferably, it is equal to or lower than 60° C.

According to this embodiment, the preferred range is 40 to 70° C., especially 40 to 60° C.

According to a second embodiment for the manufacture of fluoroethylene carbonate, the reaction temperature is preferably equal to or lower than 50° C., and more preferably, equal to or lower than 30° C. The reaction temperature is preferably equal to or greater than 2° C., more preferably, equal to or greater than 10° C., and most preferably, equal to or greater than 20° C. In this embodiment, preferred ranges are 2° C. to 50° C., more preferably 10° C. to 50° C., and especially, 20 to 30° C.

The reaction rate normally is higher at higher temperature, but the selectivity may be affected differently. Thus, it is expected that the reaction according to the first alternative which may be performed at a higher temperature than according to the second alternative, will proceed with a higher reaction rate, but with lower selectivity. Generally, it is preferred to perform the reaction according to the second alternative because the selectivity is higher, and this advantage prevails over reaction speed.

As mentioned above, the reaction temperature of the fluorination of dimethyl carbonate can advantageously be lower. Preferably, it is higher than 2° C. and equal to or lower than 50° C. More preferably, for the manufacture of fluoromethyl methyl carbonate, it is higher than 2° C. and lower than 40° C.

In the case of the fluorination of dimethyl carbonate, it may be of advantage to perform the reaction at a lower temperature level because fluoromethyl methyl carbonate has a relatively low boiling point and could leave the reaction mixture in the gas phase.

It should be noted that the fluorination reaction is performed in liquid phase (F₂ is introduced as a gas, of course). If no solvent is applied, the temperature at the start of the reaction may be in the upper range to make sure that liquid carbonate starting material is present in the reactor. When the reaction is running, fluorosubstituted carbonates function as solvent, and the reaction temperature can be lowered.

While often in chemical reactions, a 100% conversion of the starting material is aimed at, this is not the case in the frame of the present invention what concerns the carbonate starting material; for safety reasons, it is highly preferred that the fluorine is completely consumed during the reaction.

A high conversion causes a lower selectivity because of consecutive fluorination steps. A carbonate conversion, i.e. the total conversion of the carbonate in all reactors of the applied cascade in the range of 10 to 70 mol-% is preferred. A conversion of equal to or greater than 17 mol-% of ethylene carbonate or dimethyl carbonate is more preferred. A conversion of equal to or more than 20 mol-% of ethylene carbonate or dimethyl carbonate is especially preferred. The conversion might be lower than 10 mol-% but then, the process is less efficient because a lot of starting material must be recycled. For selectivity reasons, an overall conversion of equal to or lower than 65 mol-% of ethylene carbonate or dimethyl carbonate is preferred. A conversion of equal to or lower than 60 mol-% of ethylene carbonate or dimethyl carbonate is more preferred. The conversion might be even higher than 70%, but then, too much overfluorinated products are formed, and the yield is reduced significantly. A highly preferred range for conversion of the carbonate starting material is 25 to 55 mol-%.

As to the manufacture of fluoromethyl methyl carbonate, an overall conversion of equal to or lower than 40 mol-% is preferred.

Accordingly, the molar ratio of F₂ in the gaseous mixture and ethylene carbonate or dimethyl carbonate is adapted to the desired conversion taking into consideration that elemental fluorine should be totally consumed during the reaction.

In a preferred embodiment, the reaction is started using a liquid medium which consists of the ethylene carbonate starting material, optionally dissolved in fluoroethylene carbonate, or which consists of dimethyl carbonate starting material, optionally dissolved in fluoromethyl methyl carbonate. When the reaction is running, the reaction medium consists essentially of unreacted starting material, the monofluorinated product, optionally difluorinated, trifluorinated and/or tetrafluorinated product, HF, unreacted F₂ and inert gas, preferably nitrogen. If the reaction is running, it is preferred to perform the reaction such that the concentration of the starting material and the monofluorinated product present in the reaction mixture are kept within a certain concentration range. This means that the concentration of these compounds is kept reasonably constant during the reaction. Preferably, when the start-up phase is terminated, the reaction is performed such the reaction mixture contains a stationary concentration of the starting ethylene carbonate and the monofluorinated ethylene carbonate, or of dimethyl carbonate and fluoromethyl methyl carbonate, respectively. This can easily be achieved by feeding constant amounts of starting material and constant amounts of fluorine in a constant molar ratio into the reaction mixture. By adjusting the amount of the carbonate or the amount of fluorine fed into the reactor, or the amount of reaction mixture withdrawn from the reactor, the concentration can be fine-tuned. “Stationary concentration” means that the concentration of starting material and reaction product within a time range remains in a range of ±10% of the average concentration within that time range. Preferably, the time range is equal to or greater than 30 minutes, more preferably, equal to or greater than 1 hour, especially preferably, equal to or greater than 2 hours. If desired, the stationary concentration can be safeguarded automically.

The advantage is, of course, that the control of the reaction is much easier, it is performed smoothly and safely, and the yield is higher.

The pressure during the reaction is generally at least so high that the carbonate starting material remains essentially in the liquid phase. It is preferred to perform the process close to the ambient pressure. Preferably, the pressure is equal to or higher than atmospheric pressure, more preferably, equal to or higher than 1.2 bar (abs.). Preferably, the pressure is equal to or lower than 10 bar (abs). More preferably, the pressure is equal to or lower than 5 bar (abs). Most preferably, the pressure corresponds to the ambient pressure. A range of 1.2 bar (abs.) to 5 bar (abs.) is preferred. When selecting the pressure, it has to be noted that the partial pressure of fluorine in the reaction mixture should not exceed a reasonable value. Thus, if the content of fluorine in the gas mixture containing the fluorine and diluent is in an upper range, the pressure should be in the lower range. On the other hand, if the content of fluorine in the fluorine/diluent gas mixture is in the lower range, the pressure may be in the upper range. Of course, an effective cooling of the reaction mixture allows higher partial pressures of fluorine.

Should the disclosure of any patents, patent applications, and publications which are incorporated herein by reference conflict with the description of the present application to the extent that it may render a term unclear, the present description shall take precedence.

The invention will now be explained in view of preferred embodiments which provide a cascade with two and three reactors, respectively.

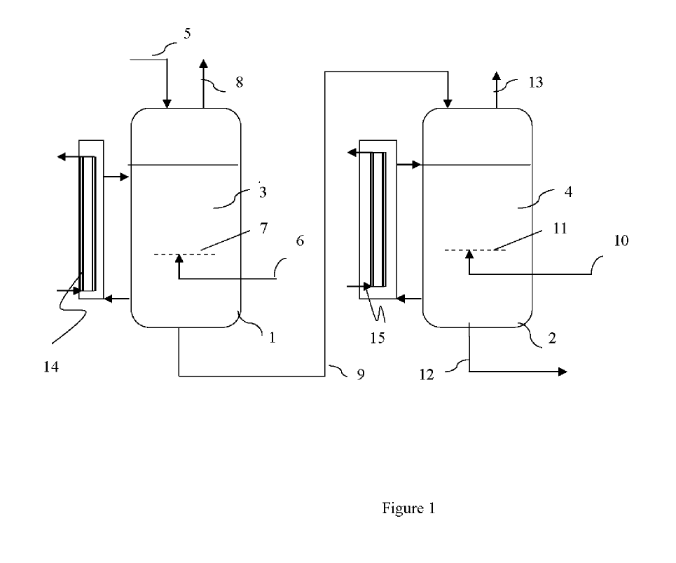

FIG. 1 shows a 2-reactor cascade in which the process of the invention can be performed. The cascade comprises 2 reactors 1 and 2 containing liquid reaction mixtures 3 and 4, respectively. Ethylene carbonate is introduced via line 5 into reactor 1. A fluorine/nitrogen mixture is introduced via line 6 into reactor 1. The gas mixture is dispersed into very small bubbles by means of frit 7. Gaseous products, mainly nitrogen (and/or another inert gas, if applied) from the fluorine/nitrogen mixture, and HF, leave the reactor 1 via line 8. The gases can be treated to remove HF and other fluorinated compounds entrained by the gas flow. For example, HF can be removed in a washer or scrubber by contact with water or acidic or basic aqueous solutions, e.g. sodium lye. It is also possible to apply a water scrubber and then to apply a washer with a basic or acidic solution. Continuously, liquid reaction mixture is withdrawn from reactor 1 through line 9 and is introduced into reactor 2 where it is once again contacted with fluorine gas (or a mixture containing fluorine gas and inert gas, especially N₂) introduced via line 10 and dispersing frit 11. Gaseous products (mainly nitrogen or other inert gas and HF) leave reactor 2 via line 13. Reaction mixture is continuously withdrawn from reactor 2 via line 12.

The cooling units 14 and 15 are operated with a cooling medium, e.g. water. The reaction mixture is circulated through cooling units 14 and 15 and cooled therein.

The reaction product withdrawn from reactor 2 via line 12 is then further processed to isolate the desired reaction product.

The isolation can be performed in any desired manner. If desired, HF can be removed by stripping from the crude reaction mixture as described in unpublished international patent application PCT/EP 2009/053561 by passing hot inert gas, especially nitrogen, through the hot or heated reaction mixture, and pure product can be obtained in a subsequent distillation.

Generally, starting material or mixtures of starting material and fluorinated product recovered during the isolation or purification steps are recycled to the reaction. This reduces costs and is ecologically advantageous.

Fluoroethylene carbonate is the most preferred reaction product. It is produced by the reaction of ethylene carbonate and fluorine, preferably diluted with nitrogen as indicated above.

In a preferred embodiment, both the reaction process as well as the isolation of the fluorosubstituted reaction products is performed continuously.

The advantage of a continuous process is that it is possible to reduce “down” times of the reactor because the reactor must not be stopped in the beginning and the end of every batch. The process is easy to program.

### EXAMPLES

The following example describes the invention in detail without intention to limit it.

### Example 1

Preparation of fluoroethylene carbonate in a 2-reactor cascade

The apparatus corresponds to the reactor shown in FIG. 1 comprising 2 reactors 1 and 2 in a cascade (the reference numbers correspond to those in FIG. 1). Before the reaction is started, ethylene carbonate and fluoroethylene carbonate are filled into the reactors 1 and 2 so that the resulting mixture contains about 10% by weight of fluoroethylene carbonate; of course, if desired, a respective mixture can be filled into the reactors. The initial addition of fluoroethylene carbonate serves to lower the melting point of ethylene carbonate when starting the fluorination reaction. Liquid ethylene carbonate is then continuously fed to reactor 1 via line 5. A gaseous mixture containing about 15% by volume of F₂ and the remainder to 100% by volume being N₂, is continuously introduced in the bottom of the first reactor 1 through a line 6 and a stainless steel frit 3. Very small gas bubbles are formed leading to a high contact surface between gas and liquid. The temperature in reactor 1 is kept at about 50° C. by circulating a part of the reaction mixture 3 through a cooler 14 operated with cooling water. The pressure in reactor 1, just as the pressure in reactor 2, corresponds to the ambient pressure (slightly above 1 bar (abs)). Gaseous constituents, mainly HF and N₂, are withdrawn from the gas space above the liquid reaction mixture though line 8. The gas is passed through a water scrubber to absorb HF. Nitrogen which passes the water scrubber is released to the atmosphere.

Liquid reaction mixture is continuously withdrawn from reactor 1 through line 9 and is introduced in reactor 2. A gaseous mixture containing about 15% by volume of F₂ and the remainder to 100% by volume being N₂, is continuously introduced into reactor 2 in the same way as in reactor 1. The temperature in the second reactor is kept at about 50° C. Gaseous constituents are withdrawn from the gas space of reactor 2 through a separate line 13 and are treated like the gases withdrawn from reactor 1. The reaction mixture continuously withdrawn from the bottom of reactor 2 through line 12 is first treated to remove most of the HF contained therein. This can be done in a stripping column by blowing hot N₂ through the heated reaction mixture. The stripped mixture is then distilled to isolate pure fluoroethylene carbonate.

While example 1 describes the use of a gas mixture of fluorine and nitrogen, it can be performed with a gas mixture of fluorine and any other inert gas or inert gases.

### Example 2

Manufacture of fluoroethylene carbonate in a 3-reactor cascade

Example 1 was repeated, but this time, a reactor cascade with 3 consecutive reactors is used. Into the third reactor (which is cooled, like the others, by circulating a part of the reaction mixture in a loop through a cooler), reaction mixture from the second reactor is introduced, and an F₂/N₂ gas mixture containing about 15% by volume of F₂ is introduced via a gas line and a frit to the reaction mixture of the third reactor, too. The reaction mixture continuously withdrawn from the bottom of the third reactor is treated as described in example 1 to isolate pure fluoroethylene carbonate.

For a calculation of the respective concentrations of ethylene carbonate, fluoroethylene carbonate and higher fluorinated products, the following assumptions are made:

Reaction temperature: 50° C.
Residence time in each reactor of the 2-reactor cascade: 2
Residence time in each reactor of the 3-reactoer cascade: 1.3
Concentration of fluoroethylene carbonate in ethylene carbonate introduced into the first reactor: 0%
Concentration of ethylene carbonate in the ethylene carbonate introduced into the first reactor: 100%

### Abbreviations:

EC: Ethylene carbonate
FlEC: Fluoroethylene carbonate
Trans: trans-4,5-Difluoroethylene carbonate
Cis: cis-4,5-Difluoroethylene carbonate
44: 4,4-Difluoroethylene carbonate
Sum: Total amount of difluoroethylene carbonates produced
Percentages are given in GC-%

Conversion is calculated as 100—the sum of the reaction products. In table 1, the global conversion is indicated.

The results or the calculation are assembled in table 1.

TABLE 1

| | | | | | | | |
|---|---|---|---|---|---|---|---|
| Stage | EC | F1EC | trans | cis | 44 | Sum | Conversion |

2-Reactor Cascade

| | | | | | | | |
|---|---|---|---|---|---|---|---|
| 1 | 69.5 | 26.3 | 2.6 | 1.1 | 0.5 | 4.2 | 30.5 |
| 2 | 48.2 | 41 | 6.7 | 2.9 | 1.2 | 10.8 | 51.8 |

3-Reactor Cascade

| | | | | | | | |
|---|---|---|---|---|---|---|---|
| 1 | 77.8 | 20.1 | 1.3 | 0.6 | 0.2 | 2.1 | 22.2 |
| 2 | 60.5 | 33.9 | 3.5 | 1.5 | 0.6 | 5.6 | 39.5 |
| 3 | 47 | 42.9 | 6.3 | 2.7 | 1.1 | 10.1 | 53 |

The calculation demonstrates that fluoroethylene carbonate can satisfactorily be produced in a continuous process in a 2-reactor cascade. A 3-reactor cascade is still more selective. It has to be noted that difference in selectivity of a 2-reactor cascade and a 3-reactor cascade increases with the degree of conversion of EC: the higher the predetermined conversion, the higher is the advantage of using a 3-reactor cascade (or a cascade with even more reactors).

### Example 3

Manufacture of fluoromethyl methyl carbonate

Fluoromethyl methyl carbonate can be manufactured from dimethyl carbonate and a fluorine/inert gas mixture analogously as described in examples 1 and 2. In view of the low melting point of dimethyl carbonate (2 to 4° C.), a solvent is not necessary. The reaction temperature will be kept lower than in the case of examples 1 or 2 to prevent the evaporation of fluoromethyl methyl carbonate. Thus, the temperature of the reaction mixture is kept at about 5° C.

1. A liquid phase process for the manufacture of an organic carbonate selected from the group consisting of fluoroethylene carbonate, difluoroethylene carbonate, fluoromethyl methyl carbonate and difluorinated dimethyl carbonate, comprising:
a reaction of ethylene carbonate as starting compound with diluted F₂ to produce fluoroethylene carbonate or difluoroethylene carbonate; or
a reaction of dimethyl carbonate as starting compound with diluted F₂ to produce fluoromethyl methyl carbonate or difluorinated dimethyl carbonate,
wherein the process is performed continuously.

2. The process of claim 1 wherein said diluted F₂ is applied in the form of a gas mixture with N₂.

3. The process of claim 1 wherein the ethylene carbonate or dimethyl carbonate as starting material is introduced neat into a reactor in which the reaction is performed.

4. The process of claim 2 wherein said diluted F₂ is contained in the gas mixture in an amount of from >0% by volume to 25% by volume.

5. The process of claim 1 wherein ethylene carbonate as starting compound is reacted with diluted F₂ to produce fluoroethylene carbonate.

6. The process of claim 1 wherein dimethyl carbonate as starting compound is reacted with diluted F₂ to produce fluoromethyl methyl carbonate.

7. The process of claim 5 wherein the conversion of the ethylene carbonate is from 10 to 70 mol %.

8. (canceled)

9. The process of claim 1 wherein the reaction for the production of fluoroethylene carbonate is performed at a temperature from 10 to 50° C.

10. The process of claim 1 wherein the reaction is performed at ambient pressure or at a pressure equal to or greater than ambient pressure and equal to or lower than 10 bar (absolute).

11. The process of claim 1 wherein the reaction is performed in a reactor containing a liquid reaction mixture, and wherein a part of the reaction mixture is circulated through a cooler to remove reaction heat.

12. The process of claim 1 being performed in a 2-reactor cascade, in a 3-reactor cascade, in a 4-reactor cascade, or in a 5-reactor cascade.

13. The process of claim 1 which is being performed in a single reactor with 2, 3, 4 or 5 partition plates.

14. The process of claim 1 wherein the reaction is performed in a reactor, wherein a liquid reaction mixture is withdrawn from the reactor, and wherein HF which is a reaction product and which is contained in said liquid reaction mixture is substantially removed by stripping with heated N₂.

15. The process according to claim 1 wherein isolation of the fluoroethylene carbonate, the difluoroethylene carbonate, the fluoromethyl methyl carbonate, or the difluorinated dimethyl carbonate is performed by a continuous distillation.

16. The process of claim 1 wherein the liquid phase in said process comprises no inert solvent.

17. The process of claim 6 wherein the conversion of the dimethyl carbonate is from 10 to 70 mol %.

18. The process of claim 1 wherein the reaction for the manufacture of fluoromethyl methyl carbonate is performed at a temperature higher than 2° C. and lower than 40° C.
